(19) United States
(12) Patent Application Publication
(10) Pub. No.: US 2017/0354547 A1
(43) Pub. Date: Dec. 14, 2017

(54) DEVICES, SYSTEMS AND METHODS FOR SENSING THE WELL-BEING OF A SUBJECT

(71) Applicant: Digisense Ltd., Petach Tikva (IL)

(72) Inventor: Eyall Abir, Hertzelia (IL)

(73) Assignee: Digisense Ltd., Petach Tikva (IL)

(21) Appl. No.: 15/411,593

(22) Filed: Jan. 20, 2017

Related U.S. Application Data
(63) Continuation of application No. 14/119,844, filed on Feb. 12, 2015, now abandoned.
(60) Provisional application No. 61/489,690, filed on May 25, 2011.
(60) Provisional application No. 61/550,967, filed on Oct. 25, 2011.

(51) Int. Cl. A61F 13/42; G01N 21/55; A61B 5/08

(52) U.S. Cl. CPC A61F 13/42; A61B 5/08; G01N 21/55; A61F 2013/424; G01N 2201/062

(57) ABSTRACT

A garment-mountable physiological parameter detector, comprising: an illuminator configured to illuminate in the direction of the garment; a photoresistor configured to receive light reflections from the garment; and an electronic circuit configured to detect a bodily secretion by monitoring the light reflections received by said photoresistor and identifying a temporal pattern being characteristic of a bodily secretion event.

### FIELD OF THE INVENTION

Some embodiments relate to devices, systems and methods for sensing the well-being of a subject, by detecting one or more physiological parameters.

### BACKGROUND

Various systems and devices for infant and adult incontinency monitoring have been previously proposed and implemented to monitor the condition of a diaper, bedding, adult incontinence brief and other similar articles. Today's parents also have an increased desire to instantly know when their infant's diaper is wet or soiled. Furthermore, monitoring of additional physiological parameters, such as breathing and body temperature, is also highly desired—both in homes and in hospital settings.

Common drawbacks of many existing detection systems are their size, complexity and reliability. There is still an unmet need for devices, systems and methods for monitoring physiological parameters of a subject, and optionally alerting the subject or a caregiver if a value of a parameter is beyond normal range.

### SUMMARY

There is provided, in accordance with some embodiments, a garment-mountable physiological parameter detector, comprising: an illuminator configured to illuminate in the direction of the garment; a photoresistor configured to receive light reflections from the garment; and an electronic circuit configured to detect a bodily secretion by monitoring the light reflections received by said photoresistor and identifying a temporal pattern being characteristic of a bodily secretion event.

There is further provided, in accordance with some embodiments, a system for detecting one or more physiological parameters, the system comprising: (a) a garment-mountable physiological parameter detector, comprising: an illuminator configured to illuminate in the direction of the garment, a photoresistor configured to receive light reflections from the garment, an acoustic transmitter, and an electronic circuit configured to detect a bodily secretion by monitoring the light reflections received by said photoresistor and identifying a temporal pattern being characteristic of a bodily secretion event, wherein said electronic circuit is further configured to transmit an acoustic communication signal from said acoustic transmitter upon detection of the bodily secretion, said acoustic communication signal being indicative of the bodily secretion; and (b) a receiving device configured to receive said acoustic communication signal and issue an alert indicative of the bodily secretion.

In some embodiments, the temporal pattern is a predetermined temporal pattern programmed in said electronic circuit.

In some embodiments, said garment comprises a disposable diaper.

In some embodiments, said detector is at least partially contained within a housing, wherein said illuminator and said photoresistor are visually exposed to a back side of said housing, said back side configured to be attached to the garment.

In some embodiments, said back side configured to be attached to the garment using a Velcro patch.

In some embodiments, said back side configured to be attached to the garment using an adhesive.

In some embodiments, said illuminator comprises a white-light LED (Light-Emitting Diode).

In some embodiments, said illuminator comprises red-light LED.

In some embodiments, said electronic circuit comprises a hardware LPF (Low-Pass Filter) configured to mitigate voltage fluctuations of said photoresistor.

In some embodiments, said electronic circuit is programmed with a software LPF configured to mitigate voltage fluctuations of said photoresistor.

In some embodiments, said bodily secretion which said electronic circuit is configured to detect comprises urine.

In some embodiments, said bodily secretion which said electronic circuit is configured to detect comprises feces.

In some embodiments, said photoresistor comprises an LDR (Light-Dependent Resistor).

In some embodiments, the detector further comprises a piezoelectric element, wherein said electronic circuit is further configured to detect breathing by monitoring an output of said piezoelectric element and analyzing said output in the frequency domain.

In some embodiments, said piezoelectric element comprises a PVDF (polyvinylidene fluoride) sheet.

In some embodiments, said PVDF sheet is internally attached to said back side of said housing.

In some embodiments, the detector further comprises a temperature sensor.

In some embodiments, said temperature sensor comprises a thermistor.

In some embodiments, the detector further comprises an acoustic transmitter, wherein said electronic circuit is configured to transmit an acoustic communication signal from said acoustic transmitter upon detection of the bodily secretion, said signal being indicative of the bodily secretion.

In some embodiments, the detector further comprises a buzzer, wherein said electronic circuit is configured to issue a sound alert through said buzzer upon detection of the bodily secretion.

In some embodiments, the detector further comprises a speaker, wherein said electronic circuit is configured to issue a sound alert through said speaker upon detection of the bodily secretion.

In some embodiments, said alert comprises an audible alert.

In some embodiments, said alert comprises digital data transmitted wirelessly from said receiving device to a remote device.

In some embodiments, said digital data transmitted wirelessly comprises digital data transmitted through a WAN (Wide Area Network).

In some embodiments, said WAN comprises the Internet, and said remote device comprises an Internet-enabled smart phone.

There is further provided, in accordance with some embodiments, an optical physiological parameter detector, comprising: an illuminator configured to illuminate in the direction of a garment; a photoresistor configured to change its resistance responsive to a level of light reflected from the garment; a resistance-to-voltage converter connected to said photoresistor; an analog-to-digital converter connected to said resistance-to-voltage converter; a memory comprising a detection rule set; and a microcontroller connected at least to said memory and to said analog-to-digital converter, said microcontroller being configured to apply said detection rule set to a digital signal received from said analog-to-digital converter, to detect a bodily secretion into the garment.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1A shows a characteristic layered diaper;

FIG. 1B shows a cross-sectional view of layers forming that diaper;

FIG. 2 shows a diaper equipped with a PVDF (polyvinylidene fluoride) sensor;

FIG. 3 shows an electronic circuit of the PVDF sensor;

FIG. 4 shows a block diagram of a system including the PVDF sensor;

FIG. 5 shows a raw time domain recording of the output of a charge amplifier circuit of the PVDF sensor;

FIG. 6 shows a chart of FFT (fast Fourier transform) conversion results of FIG. 5;

FIG. 7 shows a mesh manipulation of the FFT conversion results of FIG. 6;

FIG. 8 shows a raw time domain recording of the output of a charge amplifier circuit of the PVDF sensor;

FIG. 9 shows a chart of FFT conversion results of FIG. 8;

FIG. 10 shows a mesh manipulation of the FFT conversion results of FIG. 9;

FIG. 11 shows FFT conversion results of a test with an unattached PVDF sensor;

FIGS. 12A-C show graphs of output voltage as a function of time;

FIG. 13 shows a raw time domain recording of the output of a charge amplifier circuit of the PVDF sensor;

FIG. 14 shows a diaper equipped with a photoresistive sensor;

FIG. 15 shows a combined circuit diagram of three methods for detecting urine secretion and/or stool secretion;

FIG. 16 shows the results of an experiment with a photoresistive sensor coupled to a diaper;

FIG. 17 shows an active resistance-to-voltage converter (R/V) using OP AMP (operational amplifier);

FIG. 18 shows a circuit diagram of a transistor;

FIG. 19 shows a circuit diagram of a comparator;

FIG. 20 shows the results of an experiment with a temperature sensor coupled to a diaper;

FIG. 21 shows the results of another experiment with a temperature;

FIG. 22 shows a basic capacitor;

FIG. 23 shows a diaper having a decoration printed with conductive paint or ink on its outer shell;

FIG. 24 shows a block diagram of a system including a diaper capacitor and other electronics;

FIG. 25 shows readings of a capacitance meter connected to a diaper capacitor;

FIG. 26 shows a schematic illustration of an exemplary acoustic communication interface between a sensing device and a receiving device;

FIG. 27 shows a schematic illustration of an acoustic interface of a sensing system, according to some embodiments;

FIG. 28 shows schematic illustrations of visual alarms produced by a receiving device, according to some embodiments;

FIGS. 29A-B show front and back perspective views, respectively, of a detection unit;

FIG. 30 shows another electronic circuit of the PVDF sensor;

FIG. 31 shows another block diagram of a system including the PVDF sensor;

FIG. 32 shows another combined circuit diagram of three methods for detecting urine secretion and/or stool secretion;

FIG. 33 shows a circuit diagram of an exemplary 2^{nd} order low-pass filter (LPF);

FIG. 34 shows a graph of change of voltage during two simulated urination events;

FIG. 35 shows a flowchart of an exemplary software routine for breath detection; and

FIG. 36 shows a flowchart of an exemplary software routine for detecting fecal and/or urinal secretion events.

### DETAILED DESCRIPTION

Devices, systems and methods for physiological parameter sensing are disclosed herein. One or multiple sensors may be attached to, embedded in or integrally formed with a wearable garment, such as a disposable infant diaper or any other absorbent incontinence product, in order to sense one or more physiological parameters of the wearer.

These physiological parameters may include, for example, body temperature, urine secretion, stool secretion, breathing and body movement—the sensing of which may be advantageous, in particular, for unweaned infants as well as for certain individuals under medical attention. For example, parents of an unweaned infant may desire to be able to monitor such parameters and be alerted when a change occurs. As another example, a sensor detecting urine and/or stool secretion in a diaper may be used to aid in weaning an infant from diapers. The sensor may output an alert, such as an audio, visual and/or tactile alert, when urine and/or stool are detected. The alert may be aimed at the parents and/or the infant itself, thus assisting with the sometimes overwhelming task of potty training.

As mentioned, one example of a garment with which the sensor(s) may be associated is an absorbent incontinence product, such as a disposable infant diaper. Commonly, such products have a layered construction, which allows the transfer and distribution of urine to an absorbent core structure where it is locked in.

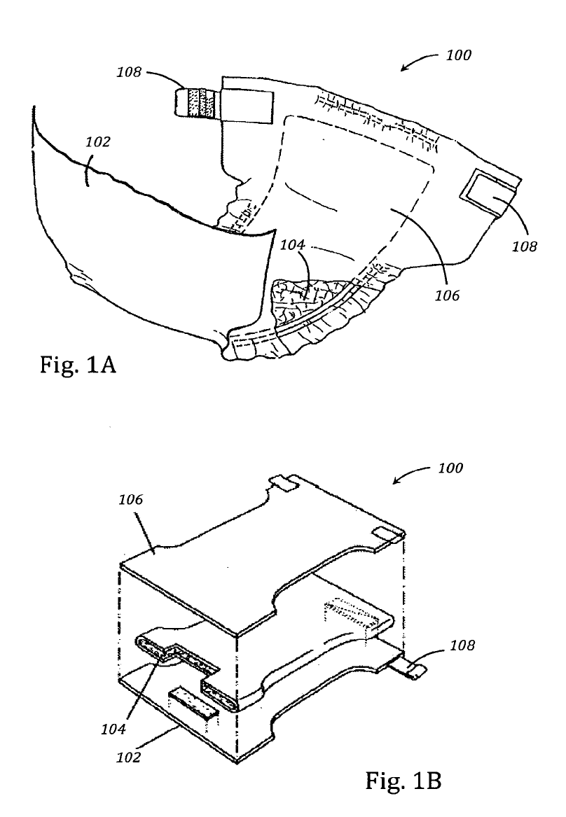

Reference is now made to FIG. 1A, which shows a characteristic layered diaper 100 in a perspective view, and to FIG. 1B, which shows a cross-sectional view of layers forming the same diaper. The basic layers found in many modern diapers, such as diaper 100, are commonly: (a) an outer shell 102, commonly made of a breathable polyethylene film or a nonwoven and film composite, which prevents wetness and soil transfer to the outside environment; (b) an inner absorbent layer 104, usually containing a mixture of air-laid paper and superabsorbent polymers; and (c) a layer 106 nearest the skin, oftentimes made of a nonwoven material with a distribution layer directly beneath, which transfers wetness to the absorbent layer. A pair of fasteners 108 is commonly used to close the diaper around the wearer's abdomen.

In some embodiments, the one or more sensors may be attached, as an external add-on, to outer shell 102 of diaper 100. In some embodiments, the one or more sensors may be embedded in or integrally formed with outer shell 102. In some embodiments, the one or more sensors may be embedded in or integrally formed with inner absorbent layer 104. In some embodiments, the one or more sensors may be embedded in or integrally formed with the layer 106 nearest the skin.

The sensing of such parameters is enabled by analyzing signals acquired by one or more sensors such as a piezoelectric sensor, a temperature sensor, a photoresistive sensor, a capacitive sensor or the like. Accordingly, embodiments discussed in the following description are arranged by the sensor types.

The term “diaper” is used here for convenience only, and may relate to any wearable garment, such as a disposable infant diaper, an adult incontinency product, underpants or the like.

Similarly, the term “infant” is also used here for convenience only, and may relate to any subject, such as a child or an adult.

### Piezoelectric Sensor

In certain embodiments, a piezoelectric transducer is used as a sensor for detecting breathing, body temperature, urine secretion, stool secretion and/or body movement.

### 1. Breathing Detection

In an embodiment, a polymeric or a crystalline piezoelectric transducer, referred to herein simply as a “piezo element”, is positioned on or in the front outer shell of the diaper, about the subject's abdomen. The piezo element then responds to the slight abdominal motion induced by the subject's breathing, and produces sufficient electrical charge for processing. An advantageous processing method, discussed below, is used for reliably detecting breath despite the lack of direct contact between the piezo element, its housing etc. and the wearer's body. Moreover, a garment, even a thick, uncondensed one such as a diaper, may physically separate between the piezo element and the wearer's body—but the processing method may nonetheless be able to reliably detect the breathing.

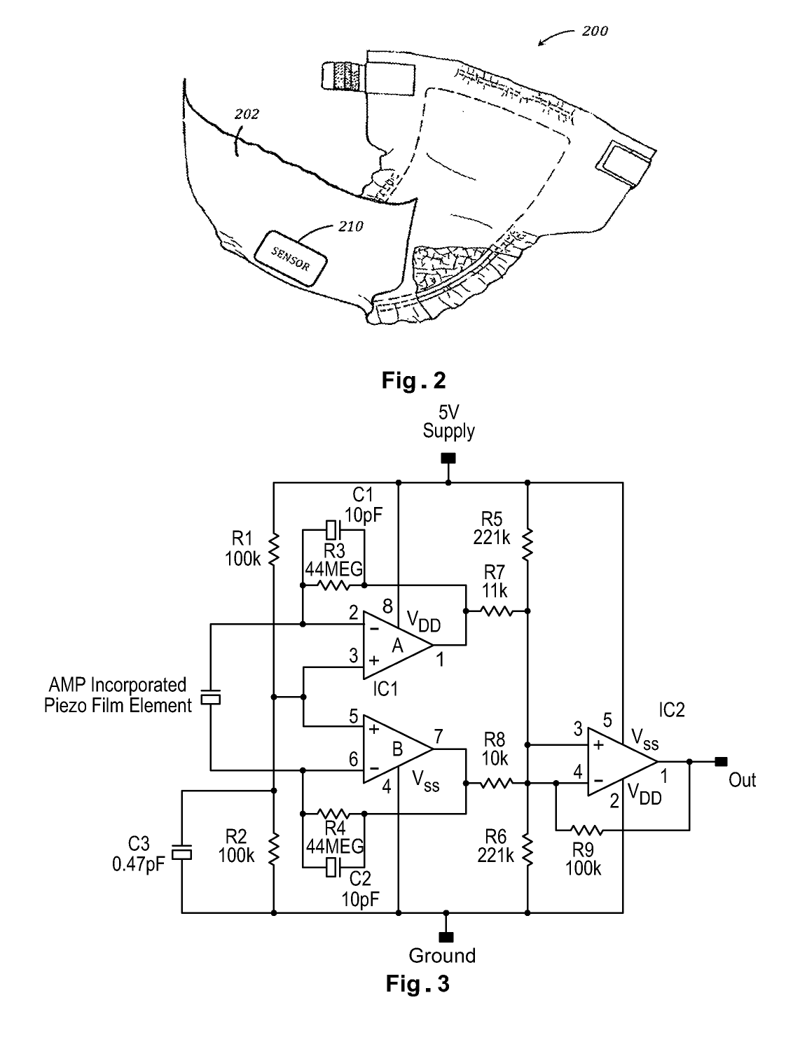

In an experiment conducted by the inventor, a one year old infant was fitted with a diaper having a PVDF (polyvinylidene fluoride) sheet-based sensor externally attached to its front part. FIG. 2 shows the configuration. A sensor 210, in this case the PVDF sensor, was attached to an outer shell 202 of a diaper 200, approximately against the infant's urethra area. A PVDF sheet manufactured by Measurement Specialties, Inc. (MEAS) of Hampton, Va., was used in the experiment.

In accordance with an embodiment, sensor 210 includes an electronic circuit connected to the PVDF sheet. One such electronic circuit is shown in FIG. 3 in detail. Those of skill in the art will recognize that the specific elements chosen to realize this electronic circuit for the experiments are meant to be demonstrative, not exhaustive. In various embodiments, one or more elements of the circuit may be replaced or omitted, and others added, as long as the mode of operation of the circuit remains similar.

The electronic circuit is, essentially, a charge amplifier circuit configured to convert the electric charge produced by the PVDF sheet responsive to the breathing motion it receives. The PVDF sheet is connected to this circuit, which is, essentially, a charge-to-voltage converter. In the experiments, the circuit was based on the low-noise Opamp IC (p/n: max4472) manufactured by Maxim Integrated Products, Inc. of Sunnyvale, Calif.

The electrical analog of the PVDF sheet is essentially a capacitor in series with a voltage source. The sheet has high output impedance and requires a high-impedance buffer amplifier. The circuit includes a differential charge amplifier (IC2) followed by a differential-to-single-ended amplifier. The differential topology reduces line-noise pickup, which is unwanted in high-gain circuits.

A dual Op Amp (IC1) endows the differential charge amplifier with single-supply operation and low supply current. Differential-to-single-ended conversion is performed by IC2. IC2, additionally, amplifies the output signal in order to bring it to a level sufficient to make good conversion to voltage.

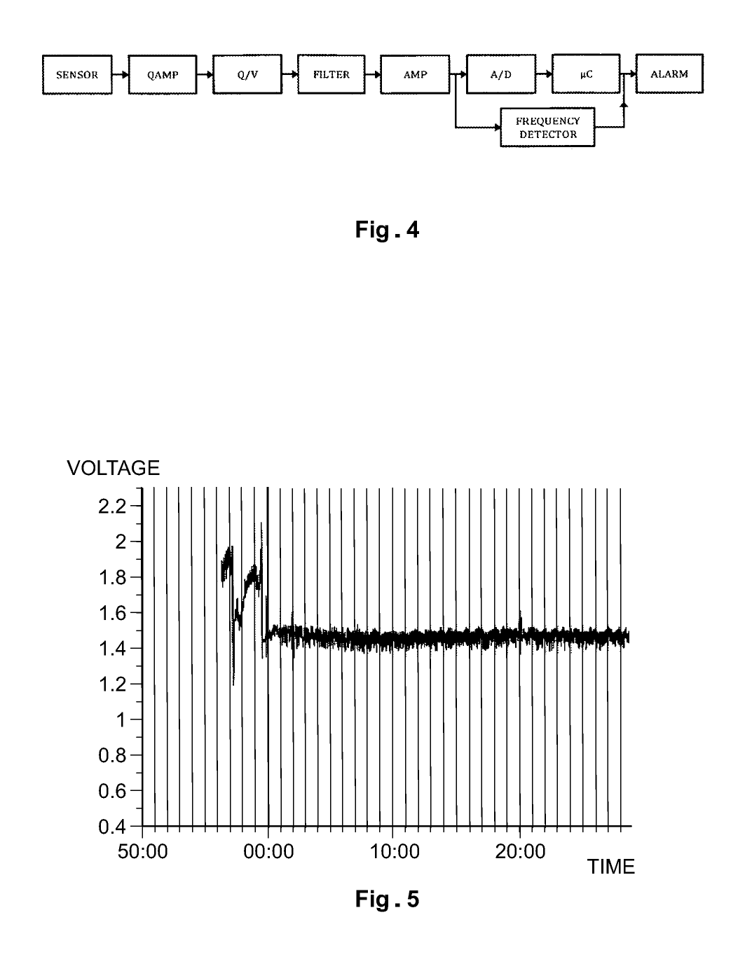

Referring to FIG. 4, the signal is next filtered by a band pass filter or a low pass filter to a desired frequency, and then amplified again. The signal is then fed into a microcontroller A/D interface.

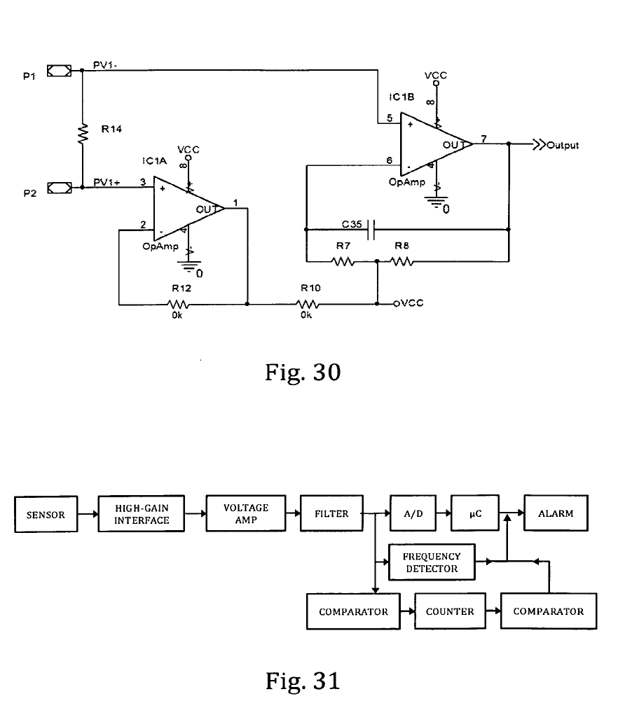

An alternative circuit is shown in FIG. 30. This circuit is, essentially, a voltage amplifier circuit configured to convert the electric voltage developed on resistor R14 by the PVDF sheet responsive to the breathing motion it receives. This circuit may be, advantageously, essentially indifferent to temperature changes. This may enhance the ability of the PVDF sheet of this circuit to sense body temperature (as discussed below) in addition to breath and/or additional parameters. In additional experiments, this circuit was based on the low-noise, low-bias, high gain Opamp IC (p/n: OP A354) manufactured by Texas Instruments, Inc.

The electrical analog of the PVDF sheet is essentially a capacitor in series with a voltage source. The sheet has high output impedance and requires a high-impedance buffer amplifier. The circuit includes a unity gain buffer (IC1) where PI and P2 are the PVDF inputs, followed by a voltage amplifier and LPF (ICIB). A very large input resistor R14 may be required to obtain a very low frequency response. For such cases, the input impedance of the buffer should be much higher than the output resistance of the PVDF sheet in order to maintain the low frequency response.

An alternative to FIG. 4, which is suitable for the FIG. 30 circuit, is shown in FIG. 31. FIG. 31 shows a hardware configuration of a comparator-counter-comparator in series, which enable the detection. These elements may be circuit-integrated or used discretely via transistor logic.

The analog signal value, which is approximately linearly correlated to the charge change of the PVDF, is sampled in a microcontroller and fed into a software routine, in order to check the charge change and determine if an alert should be issued. The alert may be relayed using a buzzer, a speaker, one or more lights and/or the like.

Two alternative embodiments of the software routine are disclosed herein. In the first, a desired preset frequency is detected. In the second, which is more sophisticated, more resource-demanding, but also more reliable and accurate, FFT (fast Fourier transform) computation is performed.

According to the second software routine, the FFT algorithm, as known in the art, is employed on the time domain signal, to detect the desired dominant breathing frequency. In the experiments, the infant's breathing rate was approximately 0.4 Hz.

In another embodiment, the analog signal value may be the input of a discrete frequency detector circuit, which determines if an alert should be issued depending on the preset of the circuit; in this embodiment, no microcontroller and software are needed.

The experimental results are as follows:

FIG. 5 shows a raw time domain recording of the charge amplifier circuit output (the artifact appearing at the very beginning of the X axis should be ignored). As can be seen easily, the voltage swings around 1.4 volt to 1.5 volt most of the time. The few sudden spikes are generated by coarse movement of the infant in bed, such turning around.

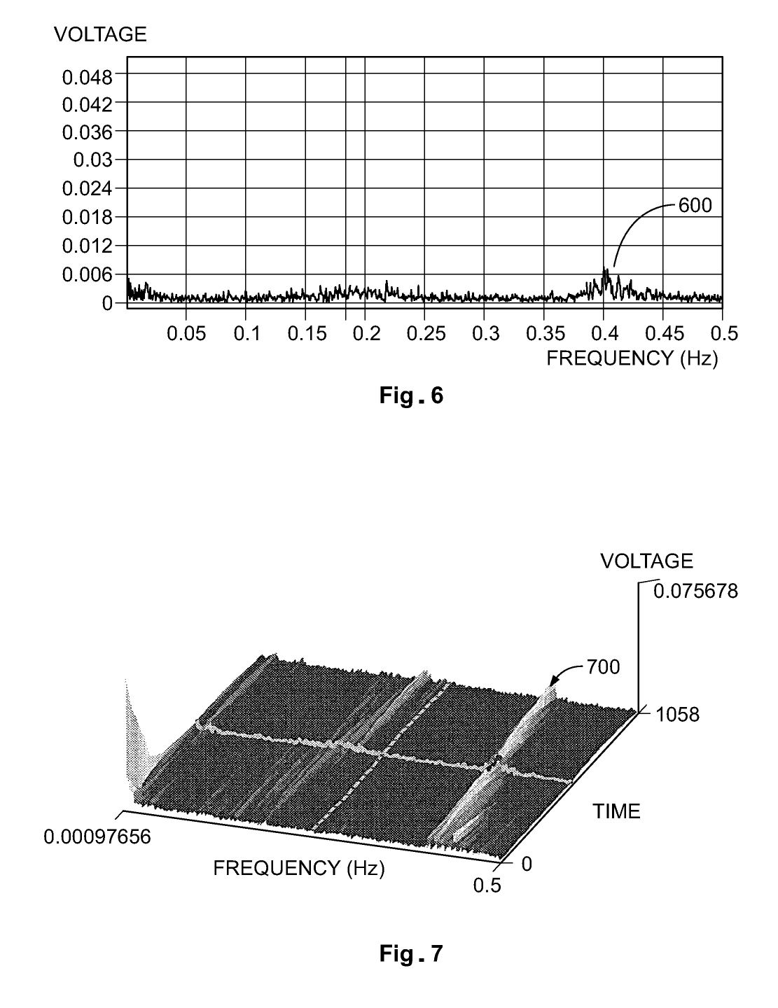

FIG. 6 shows the FFT conversion results (the artifact appearing at the very beginning of the X axis should be ignored) of the chart of FIG. 5. A dominant frequency 600 is clearly visible at 0.4 Hz, which is indeed inside the range of a normal breathing rate of infants aged 1 to 3 years (20 to 30 breaths per minute). In order to emphasize the detected dominant frequency, FIG. 7 shows it 700 in a mesh manipulation employed on the FFT chart of FIG. 6, based on 1024 temporal samples shown in the Z axis.

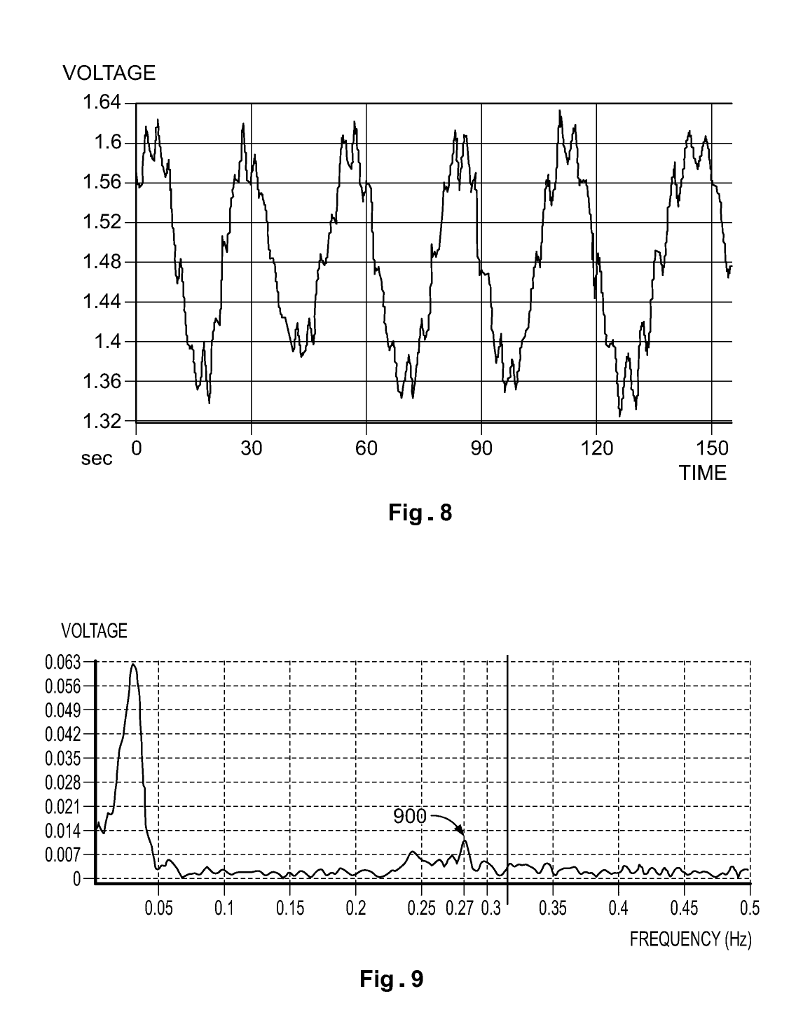

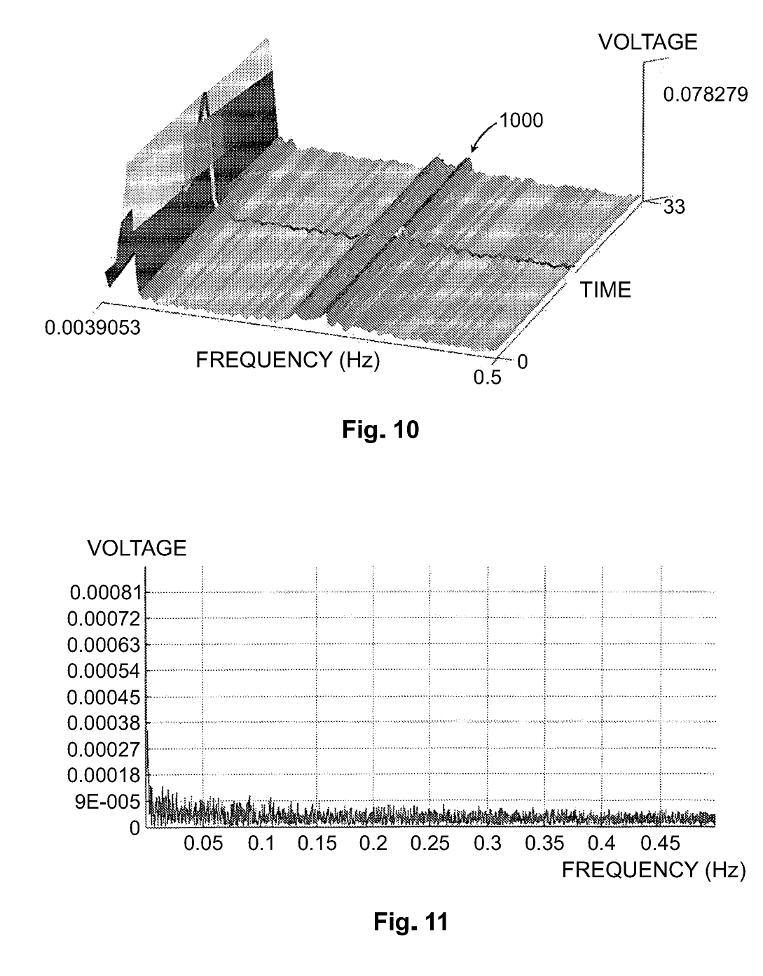

Another experiment was performed on an adult subject, age 33, in a relaxed state. The same sensor as in the former experiment was used, this time externally attached to the subject's underpants, lower stomach area. FIG. 8 shows a raw time domain recording of the charge amplifier circuit output. FIG. 9 is the FFT conversion results (artifacts prior to 0.05 Hz should be ignored) of the chart of FIG. 8. FIG. 10 is a mesh manipulation (artifacts prior to 0.05 Hz should be ignored) employed on the FFT chart of FIG. 9.

As seen in FIGS. 8-10, a dominant frequency (pointed at in 900 and 1000, respectively) appears at 0.27 Hz, which is in the range of the normal breathing rate of adults (12 to 20 breaths per minute).

For comparison, a test was conducted with a PVDF sensor which was not attached to a subject, but was rather hanging steady in the air. FIG. 11 shows FFT conversion results of this test, which lasted approximately 90 minutes. Clearly, no dominant frequency appears, in contrast to the two earlier experiments made on a human.

It was therefore proven experimentally that the present PVDF sensor (even when positioned without contacting the body, on an external surface of a garment) and software routines can successfully and reliably detect a subject's breathing, as well as its rate.

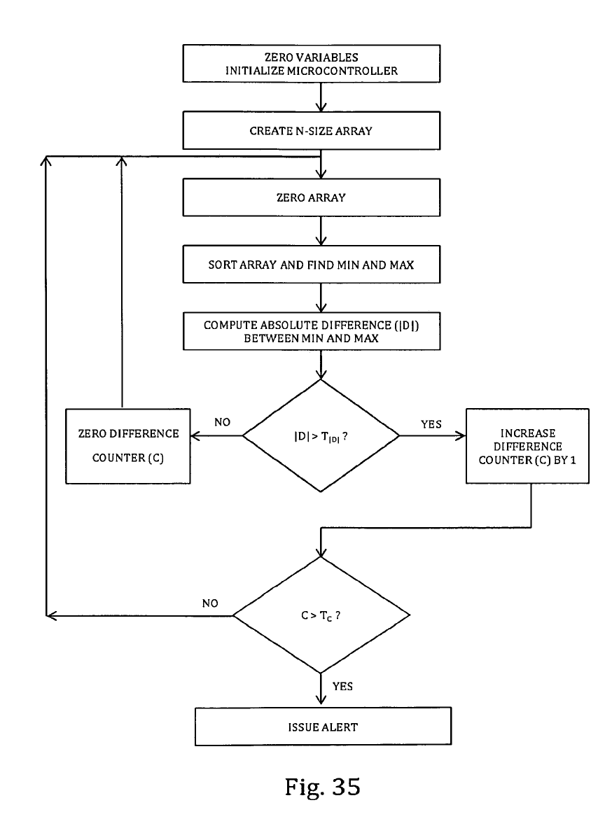

FIG. 35 shows a flow chart of an exemplary algorithm for detecting breath using a piezoelectric sensor. Initially, variables are zeroed and the microcontroller is initialized. An N-sized array is then created, zeroes, and used for storing the last N consecutive sensor readings. N may include a number of cells corresponding, for examples, to a few seconds or a few dozen seconds, based on the sampling rate of the sensor. Minimum and maximum values are then identified in the array, and the difference between them is computed. If the absolute value of the difference |D| is larger than a pre-set threshold T_{|D|}, then a difference counter C is increased by 1. If not, the difference counter C is zeroed, and the algorithm may zero the array and continue to receive sensor readings, although alternatively, the array may not be zeroed but simply continue to receive values, in a “sliding window” configuration. Each time after the difference counter C is increased by 1, the algorithm checks if the value of C has exceeded a pre-set threshold T_{c}, namely—has |D| exceeded T_{|D|} more than T_{c} times. If it did not, the algorithm may return to zeroing the array (or working in a sliding window mode, as discussed above). T_{c} is set to a value balancing between false positives and false negatives. When T_{c} is exceeded, an alert issues. T_{c} and/or T_{|D|} may be pre-programmed in the integrated circuit.

### 2. Body Temperature, Urine and Stool Detection

Another natural effect of the PVDF sheet is its pyroelectric qualities. Therefore, heat generated by the human body is absorbed by the garment or diaper, which, in turn, transfers it to the PVDF sheet. The PVDF sheet then produces different charge levels, responsive to the level of heat it absorbs.

The analog signal value is essentially linearly-correlated with temperature absorbed by the PVDF sheet. As discussed above, after the signal is sampled through the AID, it is sampled in the microcontroller and fed into the software routine in order to check the charge change and determine whether to issue an alert. The software routine detects a desired preset amplitude, which is indicative of the temperature level.

Urine and/or stool detection is also enabled by utilizing the pyroelectric characteristics of the PVDF sheet. Urine and stool secretion each cause a dramatic, rapid rise of the temperature sensed by the PVDF sensor. However, the rise caused by stool secretion may be somewhat more moderate compared to urine.

In addition, the software routine is configured to operate in a condition where the infant's body temperature is higher than normal (for example, when the infant is sick), and successfully distinguish the increased body temperature from the urine and/or stool secretion—even though the differences between the two may be barely noticeable normally.

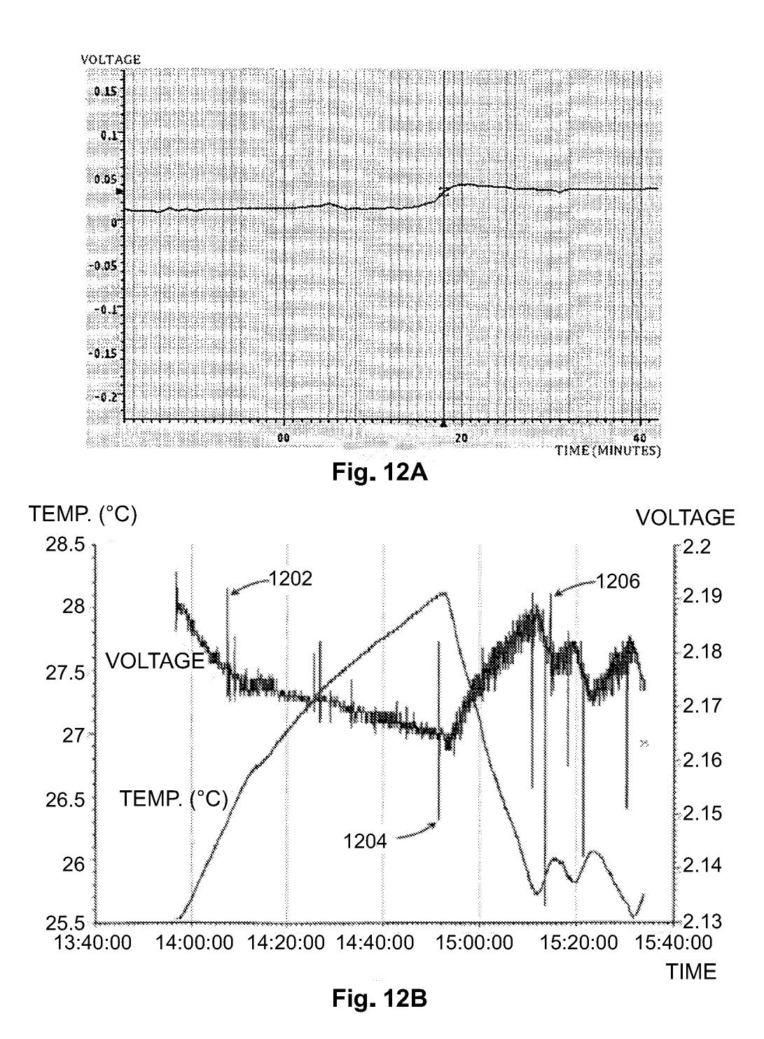

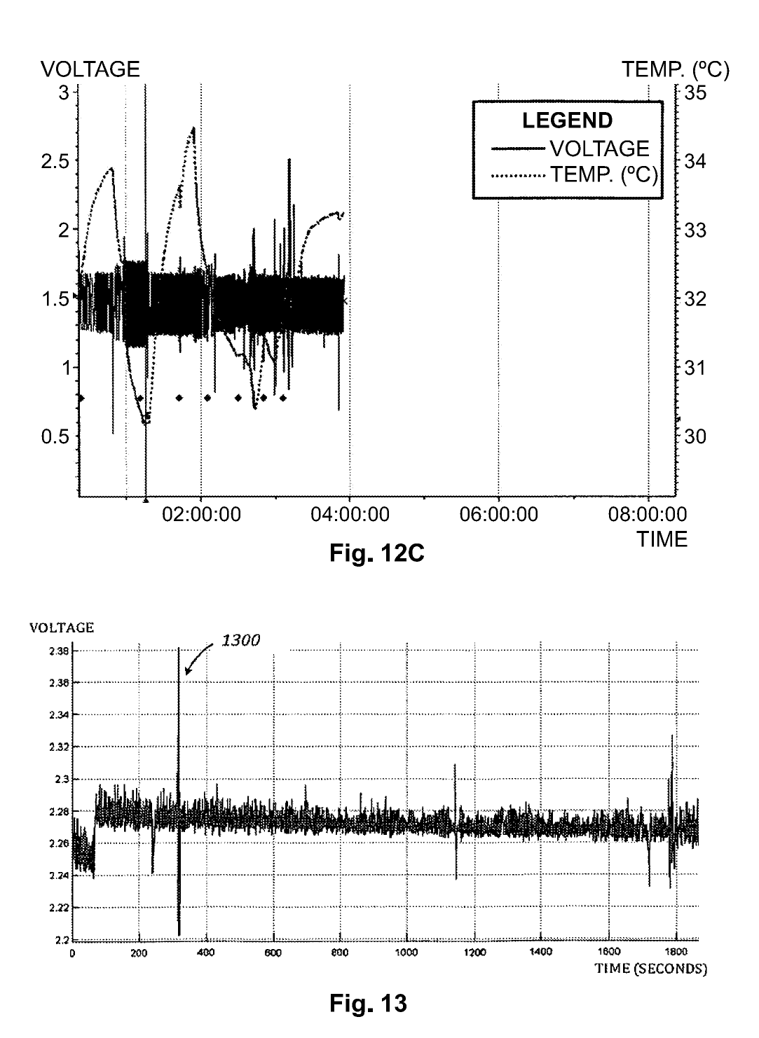

In an experiment with a one year old infant, performed similarly to the infant experiment discussed above, urine detection by the PVDF sensor was verified. FIG. 12A shows a graph of the output voltage as a function of time. Between approximately the 15^{th} and 20^{th} minutes, a clear rise of voltage is visible. Indeed, it was manually verified that the infant secreted urine at the 15^{th} minute.

Another experiment was made with a doll wearing a diaper coupled with a PVDF sensor and a type K thermistor for verification purposed. FIG. 12B shows the thermistor's reading in ° C. and the PVDF sensor's post-Q/V conversion values, over time. The two graphs exhibit inverse behavior; when the temperature rises the voltage decreases, and vice versa. The experiment was performed at a room temperature of approximately 28° C. It took the sensor about one hour to reach that temperature. Then, air conditioning has been turned in in the room, causing the temperature detected by both the PVDF sensor and the thermistor to decrease.

Another parameter whose detection was confirmed in this experiment was the PVDF sensor's sensitivity to shock, and the ability to distinguish temperature (urine, stool etc.) from shock (infant rolling in bed). Multiple shocks are visible in the voltage curve, for example, shocks 1202, 1204 and 1206, exhibited by rapid up and down surges. The shocks were simulated by knocking on the diaper. Clearly, the voltage graph allows distinguishing temperature changes from changes related to shock and movement.

Results of another experiment, performed under the same conditions, are shown in FIG. 12C. The main difference between FIG. 12B and FIG. 12C is that in the former, the signal was filtered and processed, hence exhibited a relatively small peak-to-peak amplitude; therefore, in FIG. 12B, the up/down trend of the voltage change is clearly visible as gradual increases and decreases on the Y axis (dc level). In FIG. 12C, however, the signal did not undergo any processing. Therefore, there is no Y-axis (dc level) trend readily visible. Instead, the peak-to-peak amplitude is the one demonstrating the temperature change; the larger the peak-to-peak amplitude, the lower the temperature is, and vice versa. For example, the relatively high temperature measured by the thermistor at approximately 1:50 AM is shown, in the voltage graph, as a relatively “thin” area, namely—an area having a small peak-to-peak amplitude. Similarly, the relatively low temperature measured by the thermistor at around 1:10 AM is shown, in the voltage graph, as a relatively “thick” area, namely—an area having a high peak-to-peak amplitude.

### 3. Body Movement

The PVDF sheet is also very sensitive to fast mechanical transitions, such as tilting, vibration and the like. This characteristic is utilized to sense the infant's body movements, such as rolling from back to stomach and vice versa, or making other significant movements which may indicate discomfort.

Reference is now made to FIG. 13, which shows a raw time domain recording of the charge amplifier circuit output, recorded in the same manner as in the infant experiment discussed above. A number of rapid voltage surges are visible, such as a surge 1300 at the 300^{th} second, in which the voltage rapidly changed between approximately 2.38 volts to 2.2 volts, while the steady state voltage is between about 2.27 volts to 2.30 volts. In this experiment, the infant was indeed observed rolling from back to stomach at the 300^{th} second.

In addition, the experiment whose results are shown in FIG. 12B and discussed above also verifies the PVDF sensor's ability to sense body movements.

### 4. Combined Parameter Detection

In an embodiment, a PVDF sensor may be configured to detect and distinguish between two or more of the following parameters: breathing, body temperature, urine secretion, stool secretion and body movement. Such combined detection may enable the discerning of the overall well-being of the subject. In a typical, exemplary scenario, the PVDF sensor is associated with a diaper worn by an infant. Distinguishing body temperature from urine and/or stool secretion may be done by measuring the change rate of the temperature: a relatively fast change of temperature, commonly measured over a number of minutes or dozens of minutes, is most likely the result of urine and/or stool secretion, which initially causes the temperature to rise and then drop back to the previous baseline. In contrast, an increase in the subject's body temperature, for example as a result of sickness, is usually much more gradual; in addition, if the diaper is fitted on the infant when the infant already has high fever, that high fever will be treated by the PVDF sensor as the baseline, and therefore urine and/or stool secretions may be detected normally. Even if the body temperature rises or increases while the diaper is worn, urine and/or stool secretions may be distinguished from the body temperature change by identifying smaller changes (urine and/or stool) in a longer trend (body temperature change).

Similarly, coarse movements of the infant, such as rolling over, may be distinguished from other parameters sensed by the PVDF sensor. These movements are exhibited by rapid charge surges that are very different, both amplitude-wise and time-wise, from changes caused by body temperature changes and urine/stool secretion alike.

Finally, since urine, stool, body temperature changes and coarse movements are all events which occur randomly and not at a steady frequency, performing FFT analysis on the signal acquired by the PVDF sensor may reveal the subject's breathing rate, which is usually steady—at least in relation to the other, more random, events. As mentioned, an infant's normal breathing rate is usually around 0.4 Hz, which is distinguishable, in the FFT analysis, from the other events. Even if the infant's breathing rate is different and/or changing, FFT analysis can successfully discern its rate over time.

### Photoresistive Sensor

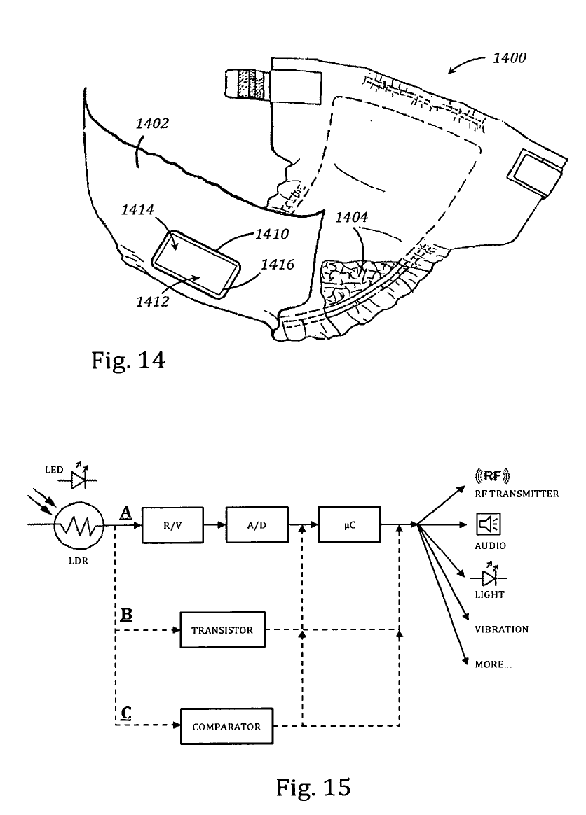

In an embodiment, a photoresistor, also referred to as a light-dependent resistor (LDR), is used for detecting urine secretion and/or stool secretion, based on the principle of a decreasing resistance when light incidence increases. With reference to a diaper 1400 shown in FIG. 14, a photoresistive sensor 1410, which includes a photoresistor 1412, an illuminator (such as a LED) 1414 and an electronic circuit 1416, may be mounted on an outer shell 1402 of the diaper. LED 1414 may be directed such that it illuminates an absorption layer 1404 of the diaper. In experiments performed by the inventor, illuminators of white light as well as of other wavelengths in the visible and invisible spectrum have been verified to be suitable. Usage of illumination may be advantageous especially in low-light conditions, such as at night, when not enough natural light is available to be reflected from the internals of the diaper.

The positioning of photoresistive sensor 1410 is shown here merely as an example. A photoresistive sensor may nonetheless be positioned in or on any of the diaper's layers; in addition, components of the photoresistive sensor may be distributed in different layers and/or in different positions.

When the diaper's absorption layer is clean, the photoresistor has steady state resistance as a result of the steady state of photons returned through the diaper by virtue of the constant illumination by the LED. As urine and/or stool are secreted into the diaper, the amount of photons reaching the photoresistor decreases, since a greater portion of the light is now blocked by the secretions. Generally, the amount of photons reaching the photoresistor changes as a function of the amount of urine and/or stool disposed inside the diaper.

Photoresistor 1412 may be positioned such that its sensing surface is directed towards the inside of the diaper, and LED 1414 may be positioned such that its light emission is also directed towards the inside of the diaper.

Electronic circuit 1416 may be a resistance-to-voltage converter. In an embodiment, further discussed below under the title “Method I”, the output resistance of photoresistor 1412 is the input of the resistance-to-voltage converter, and the output of the resistance-to-voltage converter is a voltage input to a microcontroller A/D interface.

The analog value, which is approximately linearly correlated to the resistance change of the photoresistor, is sampled in the microcontroller and fed into a software routine in order to check the resistance change in the time domain, namely—identify temporal patterns indicative of the existence of urine and/or feces, and determine if an alert is due.

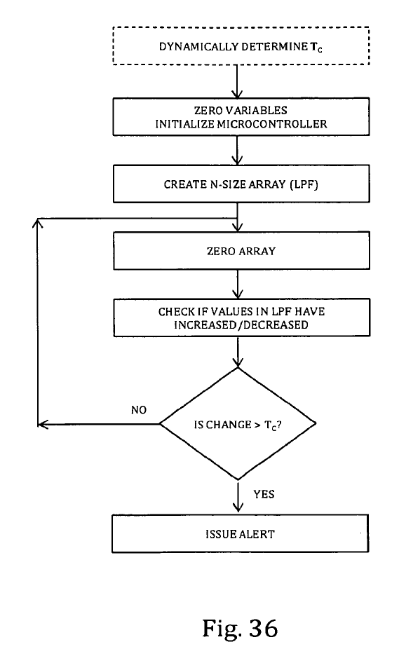

Such an exemplary software routine is shown in FIG. 36, as a flowchart. Initially, variables are zeroed and the microcontroller is initialized. An N-sized array is then created, zeroed, and used for storing the last N consecutive sensor readings. N may include a number of cells corresponding, for examples, to a few seconds or a few dozen seconds, based on the sampling rate of the sensor. The array may receive values which were already passed through a software or a hardware LPF. Then, the routine checks whether the LPF values have increased or decreased in a magnitude which exceeds a pre-set threshold T_{c} which is pre-programmed in the integrated circuit. If the threshold is exceeded, an alert is issued. If the threshold is not exceeded, the algorithm returns to zeroing the array or working in a “sliding window” mode.

T_{c} may be pre-set to match a specific type of diapers which have the same material characteristics—at least in regard to their light reflection behavior when the diaper is dry and when urine and/or feces are present. The pre-setting may be performed during manufacturing, based on tests run on the pertinent type of diaper.

Alternatively, T_{c} may be set dynamically upon each attachment to a new diaper. A calibration phase may be employed each time the sensor is turned on and/or attached to a diaper; during calibration, the initial average voltage received from the LDR is stored for later use as a baseline, namely—T_{c}.

In two other embodiments, further discussed below under the titles “Method II” and “Method III”, the output resistance of the photoresistor may also be the input of a simple transistor switch circuit, or of a voltage comparator with two voltage dividers as its inputs, respectively.

FIG. 15 shows a combined circuit diagram of three options according to methods I-III. Path A of the diagram, which may be referred to as a “digital” path, includes a microcontroller with a software decision algorithm.

Paths B and C, referred to as “discrete” paths, include a transistor or a comparator, respectively. Each of the transistor and comparator may provide an input signal to the microcontroller or, alternatively, connect directly to the notification element.

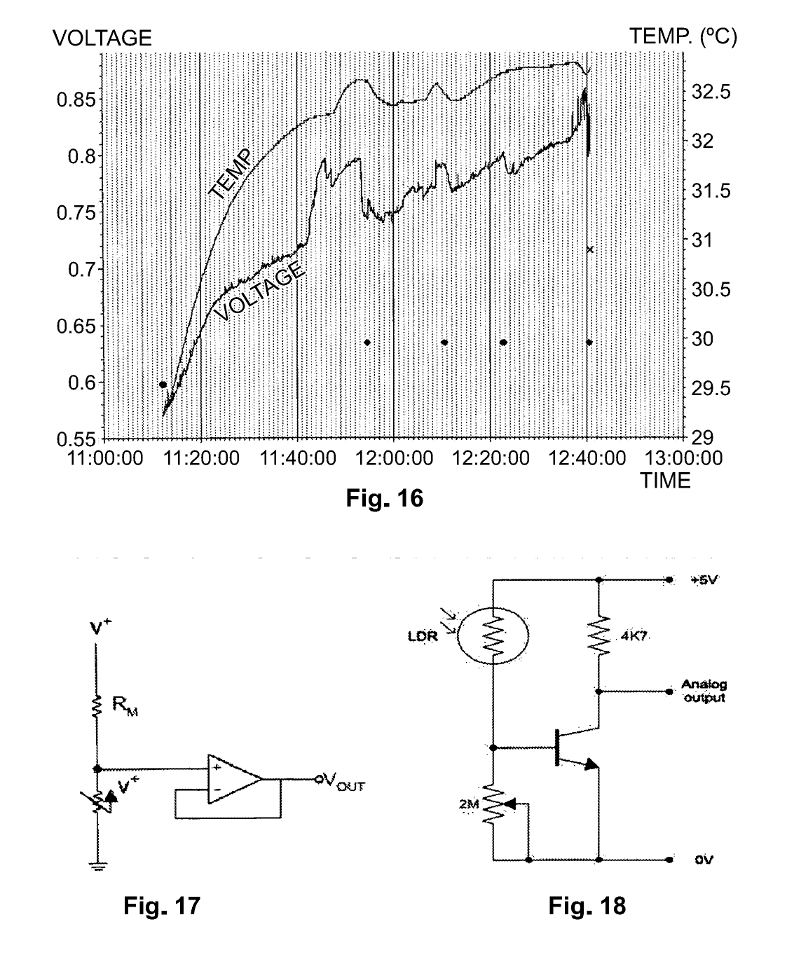

In an experiment, a diaper coupled with a photoresistive sensor, as shown in FIG. 14, was worn by a one year old infant. The results are shown in FIG. 16. The lower curve corresponds to the output from the R/V, and the upper curve is the temperature (in ° C.) inside the diaper which was simultaneously measured, using a type K thermistor, to confirm the findings.

As seen in this figure, at approximately 11:45 an increase of the voltage from about 0.71 volt to 0.8 volt has commenced. This is the result of urine disposed in the diaper's absorption layer, as confirmed manually as well as by the thermistor which showed a subsequent increase in temperature. From this point on, when urine is still disposed in the diaper, the voltage curve behaves non-linearly, unlike before. Still, it was possible to detect another urine secretion at approximately 12:10, confirmed by manual checking and temperature reading.

### 1. Method I—OpAmp

A simple active resistance-to-voltage converter (R/V) using OpAmp (operational amplifier), as shown in FIG. 17. The principle is based on a voltage divider between a fixed resistor Rₘ and a variable resistor.

The OpAmp is:
High impedance and isolator between the input voltage to the Vₒᵤt which leads to the A/D.
Unity gain.

The variable resistor shown in the figure next to an arrow is, in our case, the photoresistor. The Vₒᵤₜ voltage is connected to an A/D chip or directly to the microcontroller A/D module, if one exist, and then to one or more notification elements such a speaker, a radio transmitter, a light and/or the like.

Using the real values from the voltage graph of FIG. 16, a clear delta between the resistance with and without urine in the diaper can be observed.

Vₒᵤₜ Formula:

Vₒᵤₜ=(V+)/(l+RₘRₚₕₒₜₒ)

Rₚₕₒₜₒ calculation via the graph measurement:

Urine present: 0.792 v=3/(1+3k/Rₚₕₒₜₒ)˜Rₚₕₒₜₒ=1076 ohm

No urine present: 0.711 v=3/(l+3k/Rₚₕₒₜₒ)˜>Rₚₕₒₜₒ=931.841 ohm

Rₚₕₒₜₒ Delta=144.159 ohm

### 2. Method II—Transistor

Shown in FIG. 18, an LDR and a 2 Mohm resistor serve as a voltage divider. When light level is low (in our case, when urine exist in the diaper), the resistance of the LDR is high. This prevents current from flowing to the base of the transistor. Consequently, the output is low, commonly close to zero volts. However, when light illuminates the LDR without much interference (in our case, when urine is absent or exists in a small quantity) its resistance falls and current flows into the base of the transistor, increasing the output to about 5 volts.

### 3. Method II—Comparator

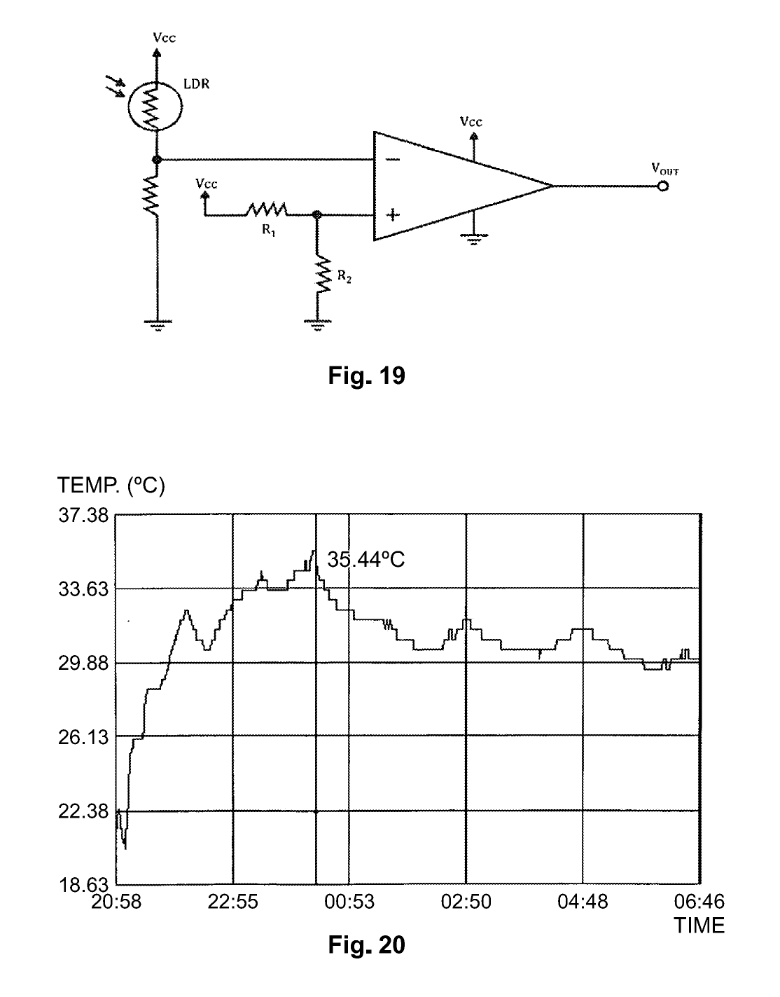

Reference is now made to FIG. 19. Resistors R₁ and R₂ are voltage dividers with a known preset level. The photoresistor and resistor R₃ are voltage dividers.

When the voltage of the negative pole of the Opamp is less than the positive pole input, then Vₒᵤₜ is HIGH. When the voltage of the positive pole of the Opamp is higher than the negative pole input, then Vₒᵤₜ is LOW.

### Temperature Sensor

In certain embodiments, a temperature sensor is used as a sensor for detecting body temperature, urine secretion and/or stool secretion. The temperature sensor may be of a type such as a resistive thermal device (RTD), Negative Temperature Coefficient resistor (NTC), Positive Temperature Coefficient resistor (PTC) or the like—all jointly referred to herein simply as a “thermistor”.

The electronics of the present temperature sensor are similar to those of the photoresistive sensor described above and shown in FIGS. 15 and 17-19, and will therefore not be repeated here. In that description and figures, the photoresistor is replaced by a thermistor, and the LED is removed. Naturally, the software routine is adapted to act on the type of output of the RTD, NTC, PTC, mutatis mutandis.

An experiment was conducted by fitting a year old infant with a diaper equipped with an NTC type sensor, positioned similarly to the photoresistive sensor experiment.

The subject fell asleep at approximately 9 PM. As shown in FIG. 20, the temperature begins to rise when the temperature sensor begins to sense the subject's body temperature. Along the night, under normal condition, the temperature stabilizes at around 30° C.

In the experiment, the infant urinated 3 times during the night, and these events were nicely detected in the signal. The first urination started at around 12 AM, which caused the temperature to gradually rise to 35.44 degrees and return to normal after a while. The second urination occurred at around 2:30 AM, and the third one at approximately 4:20 AM.

In general, the urine causes a dramatic rise of the temperature in a very short time. However, in sick infants having a relatively high body temperature, the detected temperature rise during urination may be moderate or small.

In an embodiment, such limited temperature rise is detected in the microprocessor's firmware.

Alternatively or additionally, two or more temperature sensors may be used, each in a different location. For example, one sensor at the upper front side of the diaper, for best sensitivity to body heat, and another sensor lower in or on the diaper. The software routine may then have the body heat from the upper sensor as a baseline, and detect urination based on changes in the delta between the upper sensor and the lower sensor.

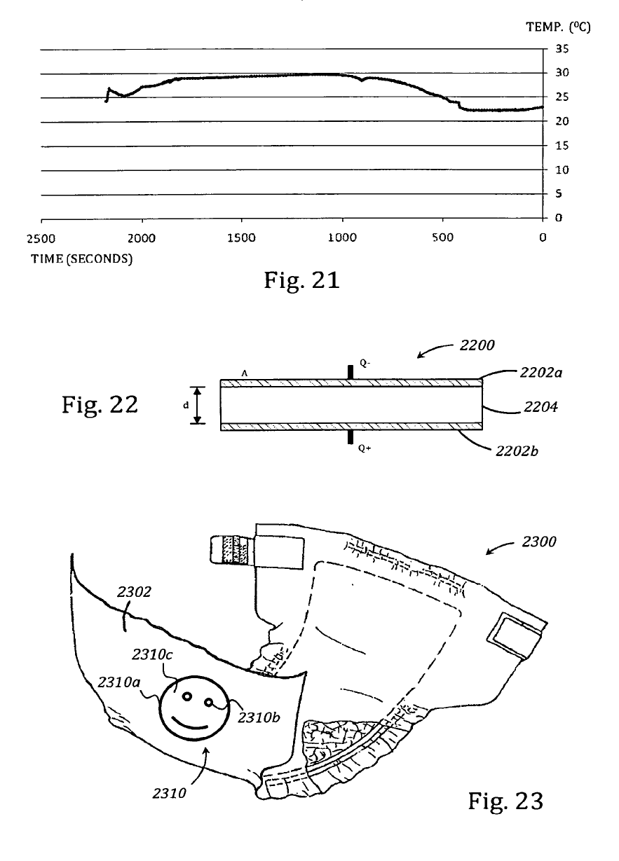

Another experiment was conducted in order to verify the above results also for stool detection. This experiment was performed on an adult. FIG. 21 shows the results. A steady state, baseline temperature of 22° C. is observed. At approximately the 500^{th} second, the temperature begins to rise towards 30° C., which is the time when, indeed, stool secretion occurred.

### Capacitive Sensor

In certain embodiments, a capacitive sensor is used as a sensor for detecting urine secretion and/or stool secretion.

Urine and stool may be considered as having the characteristics of an electrolyte solution. Hence, they can influence the electrical field in their area. FIG. 22 illustrates a capacitor 2200. Two metal conductive plates 2202a-b are separated by a dielectric material 2204.

The capacitance is given by

$$= \frac{£ o £ rA}{d} ,$$

where:

A is the area of overlap of the two plates;

£ᵣ is the relative static permittivity (sometimes called the dielectric constant) of the material between the plates (for a vacuum, £ᵣ=1);

£₀ is the electric constant (£₀≈8.854×10⁻¹² F m⁻¹); and

d is the distance between the plates.

In an embodiment, a capacitor may be implemented in the diaper as follows: The capacitor may be part of decorations printed in conductive paint, ink, and/or the like on an outer shell of a diaper.

As an example, FIG. 23 shows a diaper 2300 having a smiley face decoration 2310 printed with conductive paint or ink on the front of an outer shell 2302, Decoration 2310 is shown here only as an example. In other embodiments (not shown), the decoration may look different, have a different size and/or location. It is also possible to include multiple decorations each serving as a diaper capacitor.

Decoration 2310 implements the physics of a capacitor. Instead of metal plates 2202a-b of FIG. 22, conductive paint or ink deposits serve here as the conductors, and the dielectric material £ᵣ is the diaper's outer shell 2302 itself. For instance, an external contour 2310a of decoration 2310 may serve as a first plate, an eye 2310b of the decoration as a second plate, and an area 2310c of outer shell 2302 located between the contour and the eye serves as the dielectric material £ᵣ. Jointly, these may be referred to as a “diaper capacitor”.

In order to detect the capacitance in steady state and during change, the two “plates” of the diaper capacitor are connected to suitable electronics (not shown in the figure). The electronics are either embedded inside the diaper in the diaper manufacturing process, or attached externally—underneath the decoration or in a different location.

In both cases, the electronics may be based on a capacitance board associated with the diaper capacitor. The association may either be through a galvanic connection or a wireless connection, based, similarly, on the capacitive phenomenon. In the wireless connection, an additional capacitor, being part of the electronics, is positioned in proximity to the diaper capacitor, such as, for example, over a portion (even a very minimal one) of the decoration of the diaper. As a result, any change in the capacitance of diaper capacitor may immediately influence the capacitance of the electronics' capacitor. Practically, this means that the two capacitors are essentially electrically connected in parallel.

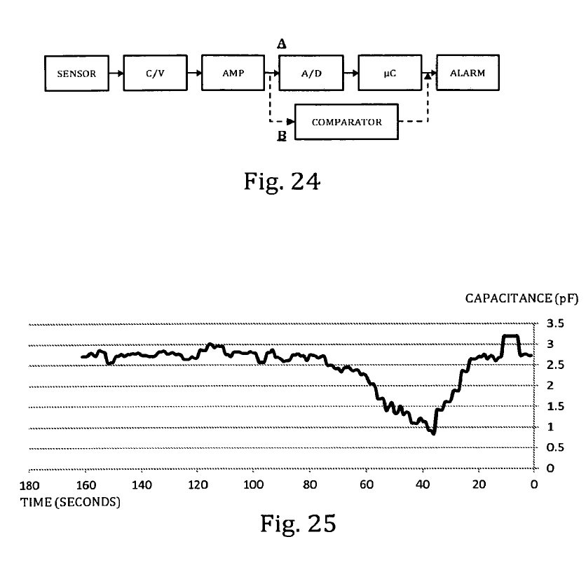

Reference is now made to FIG. 24, which shows a block diagram of a system including the diaper capacitor (referred to as “sensor” in the figure) and the other electronics.

The capacitance of the sensor is converted to voltage (C/V) in order to allow processing. The voltage signal is then amplified. In an embodiment, denoted in the figure as path A, the amplified signal is then fed into a microcontroller A/D interface. The analog signal value, which is approximately linearly correlated to the charge change of the capacitance, is sampled in the microcontroller and fed into a software routine, in order to check the charge change and determine whether to issue an alert. Preset capacitance level is defined in the software routine, and an alert is issued when this level is exceeded.

In another embodiment, denoted in the figure as path B, the analog signal value is the input of a discrete capacitance detector circuit, using a comparator. The circuit determines, based on a preset, when to issue an alert.

An experiment was performed to verify that the present capacitive sensor indeed manages to reliably detect urine and/or stool in a diaper. A smiley face was drawn on the outer shell of the diaper, similar to what is shown in FIG. 23, using a conductive adhesive tape. A second experiment utilized conductive ink, and a third utilized conductive paint.

The two smiley “plates” were connected to a capacitance meter for measurement purposes. The steady state measured capacitance was between 2.5-3 pF (pico Farad), as shown in FIG. 25. Then, an electrolytic solution was poured into the diaper, filling its absorbent layer. An immediate decrease of capacitance was then measured in the meter, as seen stating at approximately the 20^{th} second. At this point, the capacitance decreased from about 2.8 pF to about 0.9 pF. After the fluid was absorbed in the diaper, at approximately the 35^{th} second, the capacitance started climbing back to its original steady state of 2.5-3 pF.

### Acoustic Interface

According to some embodiments, the sensing devices and systems disclosed herein may further interface and/or communicate with an external and/or remote device to convey a signal generated by the sensor(s) disclosed herein to the device (herein, a “receiver” or a “receiving device”). Conveying the signal from the sensor of the sensing device to the receiving device may be performed by various communication routes, such as radio frequency or acoustic communication.

Acoustic communication makes use of sound and/or ultrasound, whereby a “transmitter” produces a sound that is detected by a “receiver”. Sound is produced by the transmitter when a physical object vibrates rapidly, disturbs nearby air molecules (or other surrounding medium) and generates compression waves that travel in all directions away from the source. Sound can be made to vary in frequency (high pitch vs. low pitch), amplitude (loudness), and periodicity (the temporal pattern of frequency and amplitude). Since acoustic waves move rapidly through the medium, acoustic signals can be quickly started, stopped, or modified to send a time-sensitive message.

According to some embodiments, for each of the various physiological conditions detected by the sensing devices and systems as disclosed herein, a different acoustical signal may be generated by one or more transducers connected to the microcontroller. The various acoustical signals may differ by various parameters, such as, but not limited to: frequency, periodicity, amplitude, duration, series of signals and the intervals there between (duty cycle) and/or the like. The frequency of the acoustic alert may be in any range. In an embodiment, the acoustic alert is in the range of 1 Hz to 10 KHz. In another embodiment, the acoustic alert is in the range of 10 Khz to 18 Khz. In another embodiment, the acoustic alert is in the range of 18 KHz to 20 Khz. In another embodiment, the acoustic alert is in the range of 18 KHz to 22 Khz. In another embodiment, the acoustic alert is in the range of 20 KHz to 22 Khz. In another embodiment, the acoustic alert is higher in the ultrasonic range, such as above 22 KHz.

For example, if the device's sensor detects urine, it may produce an 8 KHz tone, optionally in conjunction with other series of tones. For example, if the device sensor detects feces, it may produce an 8 KHz tone, optionally in conjunction with other series of tones. As another example, if the device's sensor detects high temperature it may produce a 5 Khz tone, optionally in conjunction with other series tones. As yet a further example, if the device's sensor detects a breathing problem, it may produce a 20 Khz tone, optionally in conjunction with other series of tones. These were simplistic examples, meant merely to demonstrate how acoustic communication may be realized.

According to further embodiments, the acoustical signal produced by the sensing device may be received by a receiving device, which is equipped with a microphone. Various acoustic communication protocols may be used for establishing an acoustic communication between the transmitter (the sensor) and the receiving device. For example, a publication entitled “Multi-User Frequency Hopping Underwater Acoustic Communication Protocol”, Woods Hole Oceanographic Institution, Woods Hole, Mass. 02543, May 25, 2000, the contents of which is incorporated by reference in their entirety, discloses an exemplary acoustic communication protocol in which data “packets”, similar in concept to those used in IP communications, are produced using acoustic waves.

According to some embodiments, the receiving device may include any type of device configured to receive an acoustic signal via the appropriate acoustic communication protocol, and may further convey the signal to a user, who may be located in a remote location. An added benefit of such a setting is that acoustic communication, unlike radio frequency communication, does not involve electromagnetic radiation in the subject's area, thereby increasing the safety of use of the devices and systems disclosed herein.

According to some embodiments, the acoustic tone or set of tones which may be generated by the sensing device define an acoustic protocol in the time domain. In some embodiments, the protocol may be programmed in the sensing device's microcontroller and in the receiving device.

According to some embodiments, an exemplary acoustic protocol may include the following “packets”: (1) start bit, get ready for tone sequence; (2) first tone; (3) second tone; (4) N^{th} tone; (5) stop bit, tone sequence stopped. Any of the steps and the time length, number of bits and frequency of the bit tone, loops, and the like, may be changed to define an appropriate protocol.

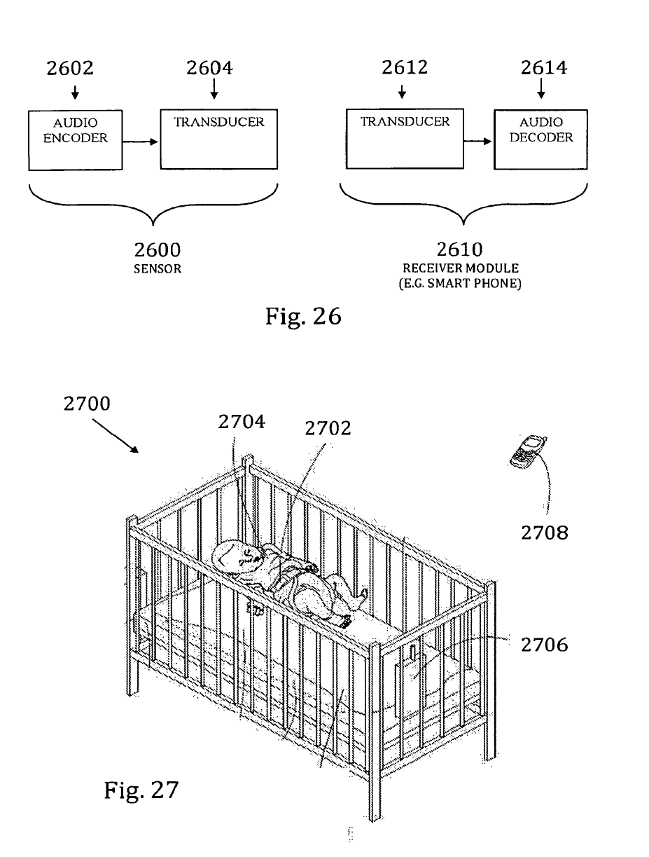

Reference is now made to FIG. 26, which schematically illustrates an exemplary acoustic communication interface between a sensing device and a receiving device. Sensing device 2600 includes an audio encoder 2602, adapted to produce an acoustic signal based on the signal produced by the sensor. Audio encoder 2602 may be incorporated in the microcontroller discussed earlier, or be connected to it. The sensing device further includes a transducing element 2604, adapted to convert an electrical signal from audio encoder 2602 into an acoustic signal transmitted towards the remote receiver. In some exemplary embodiments, the transducing element 2604 is a speaker. The acoustic signal produced by the sensing device may then be detected by transducer unit 2612 of receiving device 2610. In some exemplary embodiments, transducer 2612 is a microphone. The acoustic signal may then be decoded by audio decoder 2614 of the receiving device. Decoding the acoustic signal may be used to convert the acoustic signal to an electrical signal. The decoded signal may be processed and conveyed to a user. In some embodiments, the decoded signal may be converted to an alarm signal that may a visual signal, a tactile signal, an audible signal, and the like, or any combination thereof.

According to some embodiments, the receiving device may be portable. In some embodiments, the receiving device may be placed in the vicinity of the sensing device. In some embodiments, the receiving device may be place at a remote location, but still in acoustic communication range from the transmitting device. In some exemplary embodiments, the receiving device is a smart phone. In some exemplary embodiments, the receiving device is configured to communicate with a smart phone.

Reference is now made to FIG. 27, which schematically illustrates an acoustic interface of a sensing system, according to some embodiments. As shown in FIG. 27, in a system 2700, a sensing device 2702 is placed on a subject (exemplary baby 2704). When an event is detected by the sensor of the sensing device, an acoustic alert is produced by the sensing device. The acoustic alert is detected by a receiving device such as receiving device 2706, which is located in the proximity of the subject. The receiving device may then issue an alert (such as audible, tactile and/or visual alert) to a supervisor. Additionally or alternatively, the receiving device may serve as a relay station configured to communicate with a remote device (such as smart phone 2708), which is, in turn, configured to generate an appropriate alarm to the supervisor.

In some embodiments, the receiving device is configured to communicate with the remote device via the Internet and/or via short-range radio, utilizing technologies such as WiFi, Bluetooth, SMS, cellular data communication, push notification protocol, and activate the alarm therein, in order to notify a supervisor which may be located in a remote location. The remote device may execute an application for communicating with the receiving device and to produce audible and/or visual alarm and/or tactile alarms.

In an implementation successfully experimented with by the inventor, an Apple iPhone 4 smart phone (hereinafter “iPhone”) was used as the receiving device. The iPhone's microphone picked up the acoustic signals which were emitted by the sensing device from a range of approximately 10 meters with no walls in between or from a lower range if walls existed, and then transmitted an alert via Apple's push notification service (APN) to another iPhone acting as the remote device.

The Apple Push Notification service is intended to relay messages to iDevices even when a target application on the receiving device is not running. The APN transports and routes a notification from a given provider to a given device. A notification is a short message consisting of two major pieces of data: the device token and the payload. The device token contains information that enables the APN to locate the device on which the client application is installed. The APN also uses it to authenticate the routing of a notification. The payload is a JSON-defined property list that specifies how the user of an application on a device is to be alerted. The flow of remote-notification data is one-way. The provider composes a notification package that includes the device token for a client application and the payload. The provider sends the notification to the APN which, in turn, pushes the notification to the device.

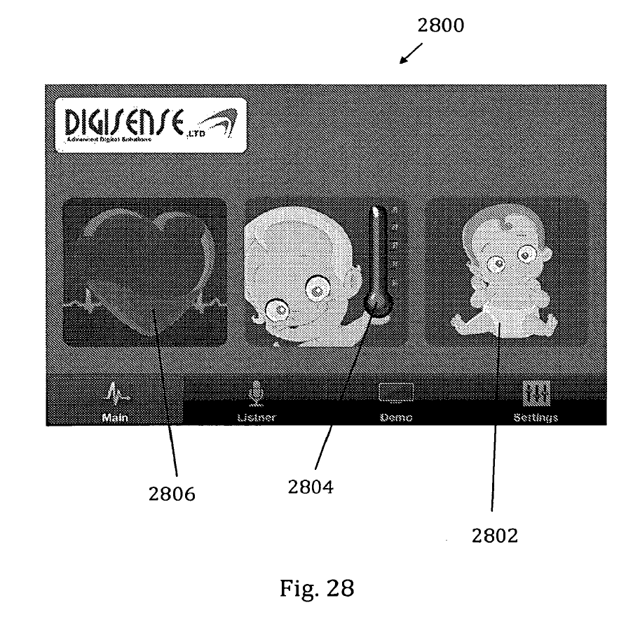

The message received by the iPhone acting as the remote device included information describing the event detected by the sensing device. The user was provided with the option of triggering the opening of the inventor's mobile application installed on this iPhone, which displayed the alert more visually. The application produced an audible, a tactile and/or a visual alarm. In some embodiments, the visual alarm is a graphical animation (shown for example in FIG. 28). For example, if urine is detected by the sensing device, an appropriate acoustic signal is generated. The acoustic signal is received by the receiving device (such as, for example, a smart phone, 2800) in which a visual and audio alarms, in the form of an animation of a diaper, changes its color to blue (2802), in conjunction with water/splash sound are produced by the smart phone. Additionally or alternatively, the receiving device may relay information regarding the detection to the remote device, in which a visual and audio alarms are activated. As another example, if feces are detected, a visual and audio alarms in the form of an animation of a diaper changes its color to brown, in conjunction with gas sounds are produced by the smart phone. For example, if high body temperature is detected by the sensing device, a visual and audio alarms in the form of an animation of a thermometer changing its color to green (2804) in conjunction with boiling sound is produced. Finally, if a breath rate problem is detected by the sensing device, a visual and audio alarm in the form of an animation of a still heart (2806) in conjunction with ambulance sound is produced.

More details on Apple's Push Notification service and related issues is available online at Apple's iOS Developer Library, http://developer.apple.com/library/ios/navigation/, which is incorporated herein by reference in its entirety.

### Examples

The sensing devices and/or systems disclosed above may be embodied, for example, as follows:

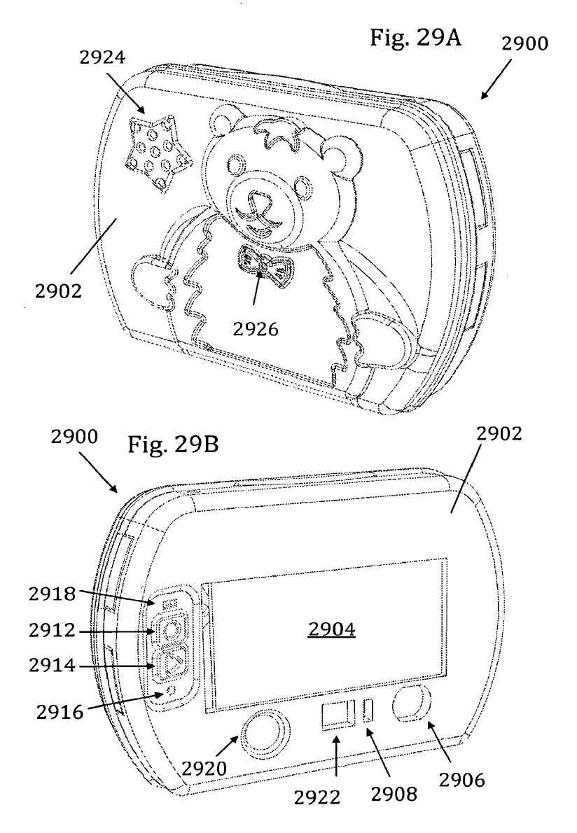

An exemplary garment-mountable physiological parameter detection unit (or simply “unit”) 2900, for use in infants and/or other persons in need thereof, is shown in FIGS. 29A-B in a front perspective view and in a back perspective view, respectively. Unit 2900 may be configured to detect one or more types of physiological parameters by sensing using one or more sensors. Examples include one or more of fecal and/or urinary secretion detection, breath monitoring, temperature measurement and/or the like.

Unit 2900 may include a housing 2902 encompassing internal components. Housing 2902 may be attached to the wearer's garment, such as a diaper, by way of a sticker, Velcro and/or the like, which may be positioned, for example, on an area 2904 or even on the entirety or part of the back side of housing 2902. Additionally or alternatively, housing 2902 may be attached to the garment using a clip and/or other means known in the art (not shown).

Unit 2900 may sense fecal and/or urinary secretions using, for example, a photoresistive sensor, as discussed above. The LDR of the photoresistive sensor may receive light through an LDR aperture 2906, and the LED of the photoresistive sensor, which is optional, may illuminate the garment through an optional LED aperture 2908. One or both apertures may be open, or closed with a cover transparent enough to allow the transfer of light. Namely—the apertures are visually-exposed at the back side of housing 2902. The cover may be, for example, the Velcro, adhesive or a different means for attaching unit 2900 to the garment. A temperature sensor, as discussed above, may be positioned at or near a temperature sensing hole 2920. Temperature sensing hole 2920 may be structured essentially as a heat sink, configured to accumulate heat for measurement by the temperature sensor. An IR transceiver may be positioned at or near an IR hole 2922.

It should be noted that some or even all sensors, transceivers, illuminators and/or the like may be positioned under the Velcro, adhesive or the different means for attaching unit 2900 to the garment, or even inside housing 2902 with no exposure to the environment. This may provide protection for the sensors/illuminators while avoiding the need to provide visible holes in housing 2902. It may also provide sealing of housing 2902, either completely or partially.

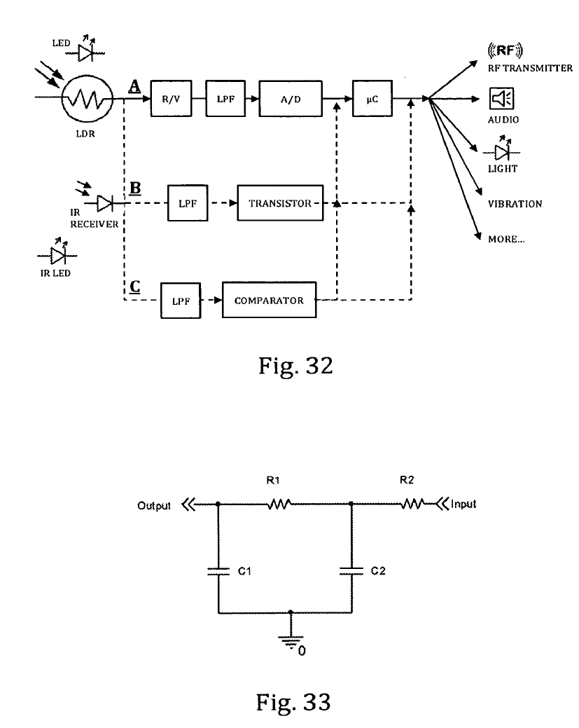

The LED may be a white-light LED, a red-light LED, an infra-red LED or a LED of a different wavelength. A white-light LED and a red-light LED have shown good results experimentally, with the red-light LED functioning especially well in low-light conditions such as in an unlit room at night. However, usage of an LDR has shown to exhibit some voltage fluctuations, as a result of the infant's movement and activity, making the detection more complex. Advantageously, software and hardware low-pass filters (LPFs), which were tested separately, were able to make the signal outputted by the LDR more parabolic and softer, making the detection of fecal and/or urinary secretion events possible using the methods and algorithms discussed here. The LPFs were based on a moving average calculation. FIG. 32 shows an alternative to FIG. 15, using LPFs. FIG. 33 shows an exemplary 2^{nd}-order LPF circuit, although those of skill in the art will recognize that an LPF of a different order and/or topology may be suitable.

In infra-red (IR) transceiver was also experimented with. As one example, a Fairchild QRE1113GR IR transceiver exhibited an uncontrolled voltage drift, causing false positive fecal and/or urinary secretion events. Since the forward voltage of the diode is temperature-dependent, as the wearer's body heats the garment (such as diaper), the voltage drift develops. This drift may be of as little as a few millivolts to a few hundred millivolts over time. Theoretically, when the garment has assumed the body's or the ambient temperatures, the voltage drift ceases. However, frequent temperature changes, although little, may continue such voltage drifts. This problem may be solved in the hardware and/or software levels. In the software level, for example, the effect of voltage drifts on true detection may be mitigated or eliminated by identifying slow voltage changes (defined as voltage change over a unit of time, ΔV/T) and disregarding them if ΔV/T is smaller than a predetermined threshold. For example, the threshold may disregard changes of 1-10 millivolts per second. Actual urination or fecal events will exceed the threshold, since they cause a rapid increase of voltage, hence causing an alarm.

If an IR transceiver is used in unit 2900, it may be provided with a suitable aperture, such as an IR aperture 2922.

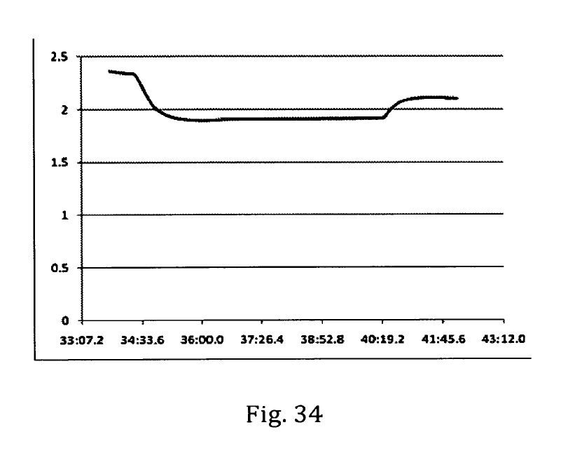

Reference is now made to FIG. 34, which shows a graph depicting experimental results with a unit such as unit 2900 of FIG. 29. The Y axis is voltage developing on the LDR and the X axis is time. No fecal and/or urinary secretion events occurred prior to minute 34, and a voltage of approximately 2.3 Volts was maintained. Then, a urine event was simulated, and the voltage decreased over less than a minute to about 1.8 Volts. During this time, the urine has been absorbed in the diaper. The unit successfully detected this event and issued an alert. The voltage remained more or less constant at 1.8 Volts until minute 40:30, when a second urine event was simulated, causing the voltage, this time, to increase to approximately 2.2 Volts. This unexpected phenomena may be dealt with, advantageously, by programming the unit to relate to increases in the absolute value of the voltage, rather than its real value.

Reference is now made back to FIGS. 29A-B. Optionally, unit 2900 includes a piezoelectric sensor (not shown) for detecting the breathing of the wearer. The piezoelectric sensor may be a PVDF sheet positioned inside or external to housing 2902, advantageously without contacting the wearer's body directly, and optionally not even through any flexible member, such as a membrane, which is sometimes used in the art for mechanically transferring motion or pressure from an object to a PVDF sheet. The PVDF sheet may be located, for example, at an area marked with phantom lines 2910, on the internal surface of the back side of housing 2902. The PVDF sheet is optionally in contact with housing 2902. Alternatively, the PVDF sheet may be mounted inside housing 2902 without contacting it. Further alternatively, a slightly depressed area in housing 2902, such as depressed area 2904, may house the PVDF sheet externally to the housing, which depressed area may be covered by the Velcro, adhesive or the like as discussed above.

Unit 2900 may further include a potty training functionality, configured to enhance the common infant potty training procedure. A parent or another caregiver may record one or more voice messages, in his or hers own voice, to be automatically played using a speaker 2924 when unit 2900 senses secretions. The voice message(s) may, for example, remind the infant that the potty should have been used, thereby providing immediate biofeedback to unintentional secretion. Optionally, to make unit 2900 more appealing to the infant, it may automatically play a pre-recorded voice message when touched by the infant.

Voice messages may be pre-recorded in the factory and/or recorded by the parents/caregivers. To that end, one or more buttons, such as a record button 2912 and/or a play button 2914 may be provided in unit 2900. Optionally, one or more status lights, such as lights 2916 and 2918, may signal to the person recording that the recording has begun, ended and/or the like. A status LED 2926 may be provided, as an example, on the front side of housing 2902, for indicating if unit 2900 is on or off, battery low indication and/or alarm light.

Optionally, either before, after or during the playing of any of such voice message, an acoustic signal may be emitted by unit 2900, to transmit an indication of the detected secretion to a remote or receiving device, as discussed above. Further optionally, the indication may trigger the loading and/or playing of an educational video clip on potty training on the remote or receiving device, which the parent and child can watch together or separately.

Further optionally, a potty may include an acoustic or a radio frequency receiver, so that when an indication of a fecal and/or urinary secretion event is received at the potty, the potty may urge the infant to use it, by emitting light, sound and/or the like. The indication may be received from unit 2900 and/or from the remote or receiving device.

In the description and claims of the application, each of the words “comprise”, “include” and “have”, and forms thereof, are not necessarily limited to members in a list with which the words may be associated.

1-46. (canceled)

47. A garment-mountable physiological parameter detector,
wherein the garment-mountable physiological parameter detector is configured to detect urine and feces in a garment and distinguish between urine and feces,
wherein the garment-mountable physiological parameter detector comprises,
an illuminator configured to illuminate in the direction of the garment, wherein the illuminator is selected from the group consisting of a white-light LED (light emitting diode), and a red-light LED;
a photoresistor configured to receive light reflections from the garment; and
an electronic circuit configured to detect a bodily secretion by monitoring the light reflections received by the photoresistor and identifying a temporal pattern being characteristic of a bodily secretion event.

48. The detector according to claim 47, wherein the temporal pattern is a predetermined temporal pattern programmed in the electronic circuit.

49. The detector according to claim 47, wherein the garment comprises a disposable diaper.

50. The detector according to claim 47, being at least partially contained within a housing, wherein the illuminator and the photoresistor are visually exposed to a back side of the housing, the back side configured to be attached to the garment.

51. The detector according to claim 50, wherein the back side configured to be attached to the garment using a Velcro patch or an adhesive.

52. The detector according to claim 47, wherein the electronic circuit comprises a hardware LPF (Low-Pass Filter) configured to mitigate voltage fluctuations of the photoresistor.

53. The detector according to claim 47, wherein the photoresistor comprises an LDR (Light-Dependent Resistor).

54. The detector according to claim 47, further comprising a piezoelectric element, wherein the electronic circuit is further configured to detect breathing by monitoring an output of the piezoelectric element and analyzing the output in the frequency domain.

55. The detector according to claim 54, wherein the piezoelectric element comprises a PVDF (polyvinylidene fluoride) sheet.

56. The detector according to claim 55, being at least partially contained within a housing, wherein the illuminator and the photoresistor are visually exposed to a back side of the housing, the back side configured to be attached to the garment,
wherein the PVDF sheet is internally attached to the back side of the housing.

57. The detector according to claim 47, further comprising an acoustic transmitter, wherein the electronic circuit is configured to transmit an acoustic communication signal from the acoustic transmitter upon detection of the bodily secretion, the signal being indicative of the bodily secretion.

58. A system for detecting one or more physiological parameters, the system comprising:
a. a garment-mountable physiological parameter detector, wherein the garment-mountable physiological parameter detector is configured to detect urine and feces in a garment and distinguish between urine and feces, wherein the garment-mountable physiological parameter detector comprises:
an illuminator configured to illuminate in the direction of the garment, wherein the illuminator is selected from the group consisting of a white-light LED (light emitting diode), and a red-light LED,
a photoresistor configured to receive light reflections from the garment, an acoustic transmitter, and
an electronic circuit configured to detect a bodily secretion by monitoring the light reflections received by the photoresistor and identifying a temporal pattern being characteristic of a bodily secretion event, wherein the electronic circuit is further configured to transmit an acoustic communication signal from the acoustic transmitter upon detection of the bodily secretion, the acoustic communication signal being indicative of the bodily secretion; and
b. a receiving device configured to receive the acoustic communication signal and issue an alert indicative of the bodily secretion.

59. The system according to claim 58, wherein the garment comprises a disposable diaper.

60. The system according to claim 58, wherein the garment-mountable physiological parameter detector is at least partially contained within a housing, wherein the illuminator and the photoresistor are visually exposed to a back side of the housing, the back side configured to be attached to the garment.

61. The system according to claim 58, wherein the electronic circuit comprises a hardware LPF (Low-Pass Filter) configured to mitigate voltage fluctuations of the photoresistor.
